(19) United States
(12) Patent Application Publication
(10) Pub. No.: US 2026/0321242 A1
(43) Pub. Date: Oct. 15, 2026

(54) SYSTEMS AND METHODS FOR SYNTHESIZING SPEECH FROM BIOSIGNALS

(71) Applicant: THE REGENTS OF THE UNIVERSITY OF CALIFORNIA, Oakland, CA (US)

(72) Inventors: David Brandman, Sacramento, CA (US); Sergey Stavisky, Oakland, CA (US); Maitreyee Wairagkar, Oakland, CA (US)

(21) Appl. No.: 19/478,214

(22) PCT Filed: Apr. 23, 2024
(86) PCT No.: PCT/US2024/025845

§ 371 Date: Oct. 23, 2025

Related U.S. Application Data
(60) Provisional application No. 63/461,507, filed on Apr. 24, 2023.

(51) Int. Cl. G10L 13/047; G10L 25/30

(52) U.S. Cl. CPC G10L 13/047; G10L 25/30

(57) ABSTRACT

Systems and methods for training a speech decoder and generating synthesized speech are provided. To train the speech decoder, systems may obtain biosignals from a subject attempting to speak a target speech sequence and generate a synthetic speech signal using a speech synthesis model. The system may then align the biosignals and the synthetic speech signal to generate a target speech signal and train the speech decoder using the biosignals and the target speech signal. To generate synthesized speech, systems may obtain biosignals from a subject attempting to speak and input the biosignals into a speech decoder to obtain a frame of an encoded speech signal. Systems may then generate a frame of a synthesized speech signal by inputting the frame of the encoded speech signal into a speech synthesizer. The frame of the synthesized speech signal may be generated within about 10 milliseconds of obtaining the biosignals.

### CROSS-REFERENCE TO RELATED APPLICATION

This application claims priority to U.S. Provisional Application No. 63/461,507, filed Apr. 24, 2023, the disclosure of which is herein incorporated by reference in its entirety for all purposes.

### BACKGROUND

Speech loss due to neurological conditions, such as amyotrophic lateral sclerosis (ALS), stroke or brain injury, as well as non-neurological conditions, such as a laryngectomy, affect quality of life and reduce independence. Enabling speech in people with anarthria is a major priority for patients and can also allow clinicians and caregivers to provide more effective care. Here, we use the term “anarthric” to refer to such individuals that cannot speak, as well as dysarthric subjects that can produce sounds, but cannot intelligibly speak compared to their neurological baseline. Current assistive communication devices (e.g., spellers with eye trackers, joystick, stylus) are slow and strenuous to use for prolonged conversations. There is a need to provide a fast and naturalistic mode of communication to millions of individuals with speech loss.

Brain-computer interfaces (BCIs) can be used to restore lost speech by decoding speech-related patterns in the neural activity recorded from the brain. Current intracortical BCIs have shown great potential for high accuracy communication by decoding neural activity related to attempted handwriting or hand movements in point and click typing in people with tetraplegia. However, current intracortical BCIs for communication using handwriting or point-and-click typing are comparatively slower than natural speech and do not capture the full expressive range of speech. Accordingly, these BCIs provide far slower modes of communication than the speed of natural speech.

In view of the foregoing, a need exists to for a new neural-decoding framework to synthesize continuous speech. One challenge in developing such a decoding framework is that for anarthric subjects, the exact details of their intended speech sequence is unknown (even in calibration protocols where the subject is specifically instructed to speak a particular phrase in a specific way). Thus, there is no readily available ground-truth to use when training the decoder. As a result, conventional approaches to producing synthesized speech are unable to produce intelligible speech for anarthric subjects, let alone doing so in a continuous manner.

The present disclosure sets forth methods and systems that address one or more of the challenges or needs mentioned above, as well as other challenges or needs.

### SUMMARY

Embodiments disclosed herein relate to training a speech decoder to restore speech to anarthric subjects. Unlike healthy patients, when a speech decoder is trained for anarthric subjects, there is no ground truth for what the anarthric subject sounds like or is trying to say.

Accordingly, techniques disclosed herein relate to synthetically generating ground truth data to train a speech decoder for anarthric subjects. Using the synthetic ground truth data, it is possible to train a causal speech decoder that can operate in a continuous manner when coupled with an anarthric subject. As a result, real-time speech capabilities can be restored to anarthric subjects.

In one embodiment, a computer-implemented method for training a speech decoder is provided. The method includes (1) obtaining, via one or more processors, one or more biosignals from a subject attempting to speak a target speech sequence; (2) generating, via the one or more processors, a synthetic speech signal representative of the target speech sequence using a speech synthesis model; (3) aligning, via the one or more processors, the one or more biosignals and the synthetic speech signal to generate a target speech signal; and (4) training, via the one or more processors, the speech decoder using the one or more biosignals and the target speech signal, wherein the speech decoder is configured to output an encoded speech signal.

In another embodiment, a computer-implemented method for generating synthesized speech is provided. The method includes (1) obtaining, via one or more processors, one or more biosignals from a subject attempting to speak; (2) inputting, via the one or more processors, the one or more biosignals into a speech decoder to obtain a frame of an encoded speech signal, wherein the speech decoder is trained using training data that includes aligned historic biosignals associated with the subject attempting to speak a target speech sequence and synthetic speech for the target speech sequence; and (3) generating, via the one or more processors, a frame of a synthesized speech signal by inputting the frame of the encoded speech signal into a speech synthesizer, wherein the speech decoder is configured to generate the frame of the synthesized speech signal within about 10 milliseconds of obtaining the one or more biosignals.

Other embodiments may include systems comprising one or more processors configured to perform the above methods and non-transitory computer-readable storage media storing instructions that, when executed by one or more processors, perform the above methods.

### BRIEF DESCRIPTION OF THE DRAWINGS

FIG. 1A schematically depicts an environment for obtaining training data to train a speech decoder.

FIG. 1B schematically depicts an environment for applying the trained speech decoder to produce synthesized speech.

FIG. 2A depicts a technique for aligning a synthetic speech signal with a neural signal of attempted speech to generate a time-aligned target synthetic speech signal.

FIG. 2B depicts additional techniques for aligning a synthetic speech signal with a neural signal of attempted speech to generate a time-aligned target synthetic speech signal.

FIG. 2C depicts a technique for aligning a synthetic speech signal with an audio signal of attempted speech to generate a time-aligned target synthetic speech signal.

FIG. 3 depicts a decoder architecture for speech synthesis from neural activity in accordance with one embodiment of the disclosed techniques.

FIG. 4A depicts an example diagram showing the encoding of neural signals into its component feature vectors to produce synthesized speech using a vocoder.

FIG. 4B depicts an example diagram showing the encoding of neural signals into kinematic signals to produce synthesized speech using a vocal tract model.

FIG. 4C depicts an example diagram depicting experimental results of a speech decoder trained in accordance with the disclosed techniques producing accurate synthesized speech in real-time.

FIG. 5 is a flowchart of an example method for training a speech decoder.

FIG. 6 is a flowchart of an example method for generating synthesized speech from biosignals.

### DETAILED DESCRIPTION

Techniques disclosed herein relate to processing one or more biosignals associated with a dysarthric or anarthric subject with a speech decoder to generate synthetic speech in a continuous manner. In some embodiments, the term “continuous” means being able to convert the biosignals to synthetic speech within perceptible delay to a subject. Generally, if the update rate for a speech decoder is greater than about 200 ms, the delay is perceptible to the subject and is less useful for restoring speech. Accordingly, in various embodiments, the update rate for converting the biosignals to synthesized speech using the speech decoders may be within about 200 ms, about 100 ms, about 50 ms, about 20 ms, about 10 ms about 5 ms, about 3 ms, about 2 ms, about 1 ms, or even less. It should be appreciated that the window size for the input to the speech decoder may be larger than the update rate.

Additionally, speech decoders trained in the disclosed manner provide a continuous output space. In other words, the speech decoder is capable of producing speech with the manner that the user is attempting to say each word (e.g., speed of speech or emphasis on a syllable of a word) which spans continuous speech output space. Similarly, the speech decoder reflects the speaker's cadence, pace and speed, which changes dynamically during speaking.

This is in contrast with some of the other approaches of speech synthesis that predict sequences of discrete “speech units” from neural activity. This discretized output speech sounds the same each time regardless of how the user tries to say each word. Additionally, discretized output speech is limited to expressions found in dictionaries of discrete outputs and generally cannot reproduce other types of vocalizations, such as non-word expressive sounds, interjections, pseudo-words that are not in the dictionary, or any other sounds used in able-bodied human speech. In sum, the ability of the speech decoder to have a continuous output space enables more natural sounding synthesized speech.

Additionally, according to certain aspects, techniques disclosed herein relate to training a speech decoder for anarthric subjects which cannot readily provide ground truth training data to train a speech decoder.

While the term “attempted” speech is generally used throughout the disclosure to refer an actual attempt to produce speech, unless otherwise stated, any such reference envisions other types of imagined or non-vocalized speech (sometimes called “mimed” speech).

Accordingly, the techniques may generally be applied to decode any speech intention or perceived speech of a subject that can be detected via the biosignals, as described herein.

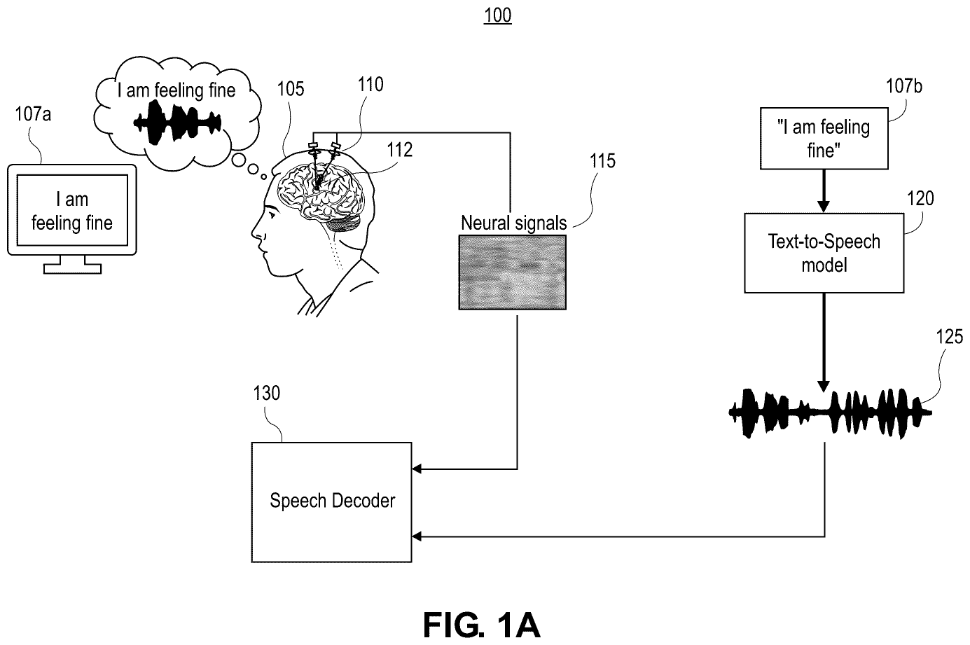

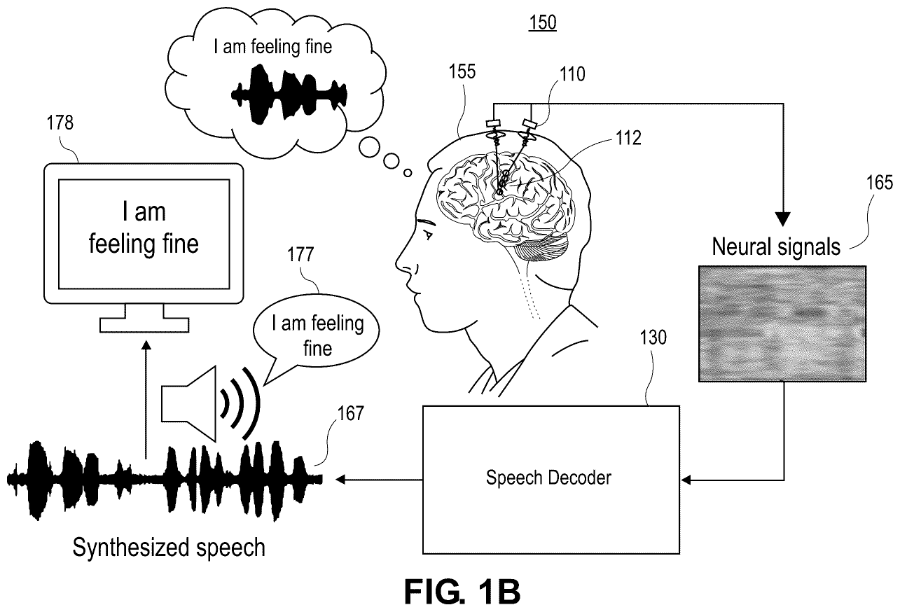

FIG. 1A depicts an example environment 100 for obtaining training data to train a speech decoder 130. As illustrated, FIG. 1A includes a BCI 105 coupled with a brain 112 of a subject 110. The BCI 105 may be coupled to the ventral motor cortex, the dorsal motor cortex, another cortical or subcortical brain area, white matter, brainstem, cerebellum, rostral cervical spine, or any other area where neural activity associated with speech may be detected. In one example, the BCI 105 includes one or more Utah arrays implanted in the ventral (orofacial) motor cortex. It should be appreciated that while FIG. 1A depicts the BCI 105, in other embodiments, other sensors may be coupled with the subject 110. For example, the subject 110 may instead be coupled with an electrocorticography (ECOG) sensor, a stereoelectroencephalography (sEEG) sensor, a surface electroencephalogram (EEG), a functional near-infrared spectroscopy (fNIRS) sensor, a magnetoencephalography (MEG) sensor, and/or other types of brain sensors.

According to aspects, a target speech sequence 107 is obtained for use when generating the training data. The target speech sequence 107 may be of a length to generate sufficient training data for training the speech decoder 130. For example, the target speech sequence 107 may correspond to several seconds of training data, over a minute of training data, over 10 minutes of training data, over 30 minutes of training data, over an hour of training data, over 90 minutes of training data, and so on.

The BCI 100 is configured to produce neural signals 115 while the subject 110 is attempting to speak a portion of the speech sequence 107a (“I am feeling fine”) as it is displayed on a screen. It should be appreciated that the word “attempting” is used because anarthric subjects are unable to intelligibly speak the target speech sequence 107a.

Accordingly, if the subject 110 is anarthric, the subject 110 may visualize or otherwise attempt the act of speaking the target speech sequence 107a to generate neural activity in a manner as if the subject 110 were actually speaking the target speech sequence 107a. In scenarios where the subject 110 is dysarthric, the attempt to speak the target speech sequence 107a may also generate audible sounds, even if the sounds do not coherently represent the target speech sequence 107a to a listener.

In some embodiments, the subject 110 may be coupled with additional or alternative sensors not coupled to brain 112. For example, the neural signals 115 associated with the kinematic motion for producing speech may be detected via an electromyography (EMG) sensor, an acoustic myography (AMG) sensor, a magnetomyography (MMG) sensor, an ultrasound sensor, a light detection and ranging (LiDAR) sensor, an infrared sensor, a motion sensor, an inertial measurement unit (IMU), a vibration sensor, a breathing sensor (e.g., a breath belt), and/or a piezoelectric sensor coupled with the larynx of the subject 110.

Additionally or alternatively, a video sensor may be configured to record kinematic data associated with facial and/or other bodily movements associated with the subject 110 while attempting to speak the target speech sequence 107a. Still further, an audio sensor may be configured to record the audio data produced by dysarthric subjects when attempting to speak the target speech sequence 107a. In view of the above, the term “biosignals” is generally used herein to refer any type of signal generated when the subject 110 is attempting to speak. While FIG. 1A only depicts the collection of the neural signals 115 from the BCI 105, in other embodiments, the neural signals 115 may be supplemented or replaced with one or more other types of biosignals described herein.

As illustrated, the environment 100 also includes a text-to-speech (TTS) model 120 configured to generate a synthetic speech signal 125 representative of the target speech sequence 107b. In some embodiments, the TTS model 120 is a synthetic speech model trained using historical speech data of the subject 110. For example, an archive of historical audio and/or video files of the subject 110 speaking before the onset of dysarthria or anarthria may be obtained. The audio data from the from the historical files may be input into a transcription model to generate the corresponding text data for the audio data spoken by the subject 110.

The audio data the transcript may then be used to train the synthetic speech model to output speech data that mimics healthy speech of the subject. Accordingly, in these embodiments, the outputs of the speech decoder 130 are able to produce synthesized speech that closely mirrors the original speech patterns of the subject 110. Thus, the resulting synthesized speech sounds close to the subject's voice.

In embodiments where historical audio data is not available, a pre-trained synthetic speech model may be chosen based on one or more characteristics of the subject 110 and/or their voice. To this end, a database of synthetic speech models (not depicted) may include models that are designed to represent individuals having different sets of characteristics (e.g., gender, age, country of origin, accent, speech style, and/or any other characteristic that can affect a person's voice). Accordingly, in these embodiments, the TTS model 120 may include the synthetic speech model that has a set of characteristics that most closely resembles the subject 110.

In some embodiments, the TTS model 120 may include a kinematic model and/or a kinetic model of articulators that assist in speech. In these embodiments, the speech is generated by replicating the kinematic and/or kinetic motion of articulators when speaking and routing the kinematic and/or kinetic data into a vocal tract model that converts the kinematic and/or kinetic data into sound. That is, rather than trying to replicate an audio output, the kinematic and/or kinetic approach attempts to replicate the articulation inputs that produce the speech in an attempt to create speech more accurately. In these embodiments, the kinematic and/or kinetic model and/or vocal tract model may by tuned to the subject 110 using a 3-D scan of the subject 110 to align characteristic(s) of the model to the anatomy of the subject 110.

Due to the differences in how the signals are generated, the target speech sequence 107 manifests differently in the neural signal 115 and the synthetic speech signal 125. That is, time at which a given word is represented in the neural signal 115 is generally different from the time at which the given word is represented in the synthetic speech signal 125. Additionally, the duration of the representations between the neural signal 115 and the synthetic speech signal 125 may vary. Thus, to generate the training data for the speech decoder 130, the portions of the neural signal 115 and the synthetic speech signal 125 that correspond to the same segment of the target speech sequence may need to be mapped to each other and/or aligned to the same duration. More particularly, the synthetic speech signal 125 may be aligned with characteristics extracted from the neural signal 115.

In some embodiments, the speech is segmentized by syllable. Segmenting using syllable as the unit of speech enables the control of prosody in the synthetic speech signal 125. In other embodiments, other units of speech may be used to segment the signals 115, 125. For example, in other embodiments, the signals 115, 125 may be segmented by word or phoneme.

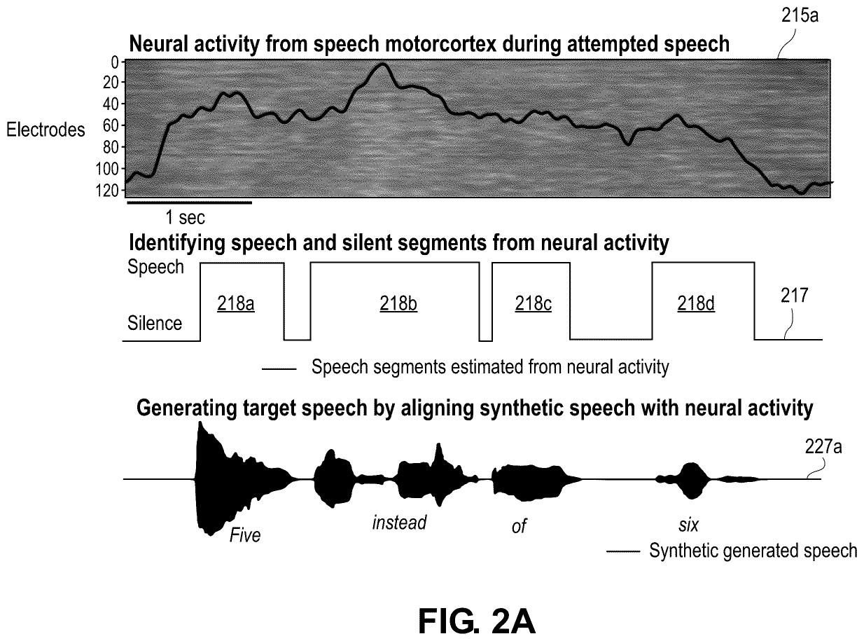

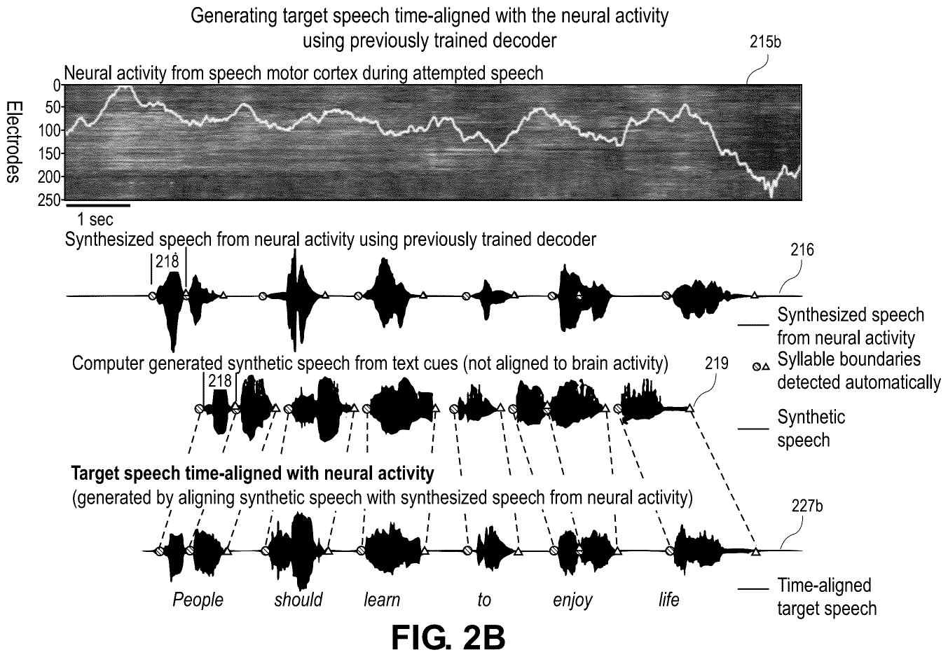

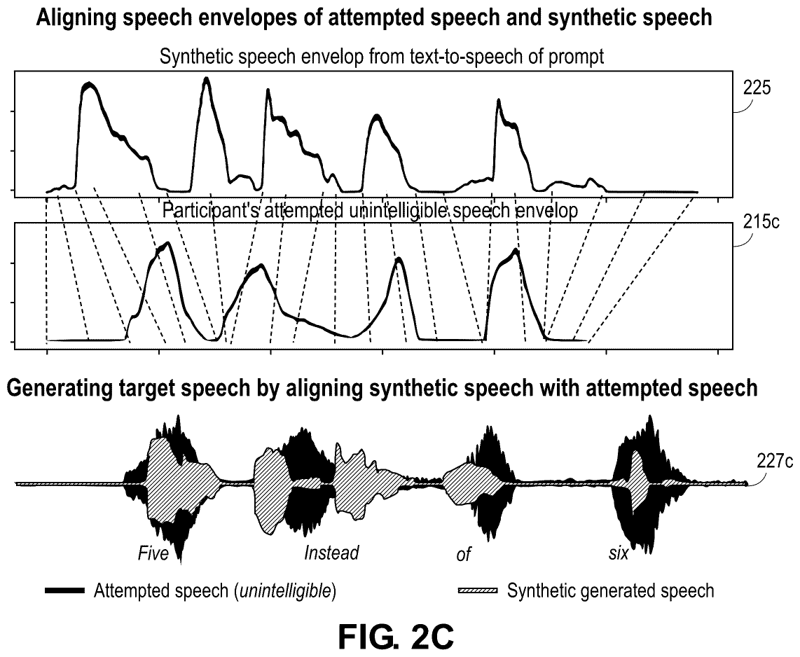

With reference to FIG. 2A, illustrated is a technique for segmenting a neural signal 215a (such as a neural signal 115). The illustrated neural signal 215a represent the neural activity of a subject speaking the expression “Five instead of six.” As illustrated, the neural activity levels in the neural signal 215a includes dynamics (e.g., plateaus and valleys) that roughly correspond to the speech segments and the silence periods therebetween.

In some embodiments, the neural signal 215 may be generated based on extracted spike-band power from neural signals recorded from each electrode of a BCI (such as the BCI 105) with a sampling rate of 30 kHz, acquired using a Blackrock Neurotech NeuroPort system. The raw neural signals may then be first filtered between 250-5000 Hz using a non-causal, fourth order Butterworth filter followed by common average referencing or linear regression referencing within each array. The spike-band power may then be computed by squaring these filtered voltage time series and binning the spike-band power by averaging over non-overlapping 10 ms sliding windows. The resulting bins may then be normalized the data by subtracting the mean and standard deviation from prior experimental results, log-transformed, and smoothed. It should be appreciated that the foregoing is one example way to generate a neural signal from a BCI. Other types of BCIs and/or other sampling techniques may be implemented in other embodiments.

Accordingly, systems disclosed herein may include analyzing the neural signal 215a to identify these peaks and valleys to produce a speech segmentation signal 217 that includes on-periods 218 that correspond to the component speech segments of a target speech sequence. After generating the speech segmentation signal 217, the systems may then assign each on-period a corresponding portion of the target speech sequence. In the illustrated example, the on-period 218a corresponds to “Five,” the on-period 218b corresponds to “instead,” the on-period 218c corresponds to “of,” and the on period 218d corresponds to “six.” The systems may then segment the neural signal 215a using the speech segmentation signal 217 to associate the portions of the target speech sequence with the corresponding portions of the neural signal 215a. It should be appreciated that while FIG. 2A depicts producing the speech segmentation signal 217 that segments the speech into on-periods 218 that correspond to words, in other embodiments, the on-periods 218 may correspond to syllables or phonemes. For example, in embodiments with syllable-wise segmentation, the on-period 218b may instead be divided into two component on-periods for each syllable of the word “instead.”

In other approaches, the speech decoder 130 is further trained to predict the text represented by the neural signal 215a. In the example illustrated in FIG. 2A, this would include analyzing the neural signal 215a to identify that the subject intended to speak the phrase “five instead of six.” Based on the predicted text, the disclosed systems may then identify the portions of the neural signal 215a that align with each specific unit of segmentation.

Accordingly, the speech segmentation signal 217 may include periods 218 that correspond to the portions of the neural signal 215a assigned to each unit of segmentation.

In still other approaches, a time-series prediction model, such as a Hidden Markov Model (HMM) or a time-series with connectionist temporal classification (CTC) model, is used to segment the neural signal 215a. More particularly, the neural signal 215a may be input into the time-series prediction model to predict when each speech segment was spoken. Accordingly, the speech segmentation signal 217 may include periods 218 that correspond to the portions of the neural signal 215a identified by the time-series prediction model. In some further approaches, the periods 218 are defined via a combination of any of the foregoing approaches (e.g., by averaging the outputs of the combined approaches).

FIG. 2B depicts another approach to segmentation to produce a time-aligned target speech signal 227b. In one illustrated approach, a speech decoder model may be used to predict the output speech based on the neural signal 215b (such as the neural signal 130).

While prior speech decoder models exhibit the drawbacks described herein, prior speech decoder models may still provide sufficient accuracy for segmenting the neural signals into the corresponding speech segments. In this approach, an output speech signal 216 may be analyzed to define the periods 218 that correspond to each unit of segmentation (e.g., word, syllable, phoneme, etc.). In the illustrated example, segmentation is performed using syllable-based segmentation. Accordingly, the output speech signal is analyzed to define the inter-syllable boundaries that define the segments 218.

Regardless of the segmentation technique, the systems may also generate a synthetic speech signal 219 (such as the synthetic speech signal 125) and identify and extract the corresponding segments of the target speech sequence in the synthetic speech signal 219. As described above, the segment of the synthetic speech signal 219 that represents the portion of the target speech sequence may vary from the duration in the segmented neural signals 215.

Accordingly, the systems may apply a time-warping function to the synthetic speech signal 219 such that the extracted segments of the synthetic speech signal 219 are aligned in duration in the time domain with the corresponding segments derived from the neural signal 215a, b.

The systems may repeat the process of mapping the corresponding portions of the neural signals 215 and the synthetic speech signal to one another and performing a time-warping function to the segment of the synthetic speech signal until each component segment of the target speech sequence is aligned with a segment of the neural signal 215. The systems may then combine the aligned signals to produce a target synthetic speech signal 227a or 227b. As illustrated, when combining the component segments, the systems may separate the component segments using a duration of the corresponding off-period and/or define segments corresponding to silent periods in the speech segmentation process. As a result, the target speech signals 227 generally reflect a speech pattern if the subject were to be able to actually speak the target speech sequence (e.g., by matching the subject's cadence, pace, and speed). Moreover, because the mapping of the neural signals 215 reflect the kinematics of speech articulators, a speech decoder trained in the described manner able to avoid false activations when the subject is silent or listening to other people speak.

In some embodiments, in addition to temporal alignment between the neural signals 215 and the target synthetic speech signals 227, the techniques may decode and align para-linguistic characteristics of the speech represented by the neural signals 215. For example, the techniques may detect pitch, intonation, prosody, and/or volume of the speech represented by the neural signals 215 and adjust the target synthetic speech signals 227 in accordance therewith. As a result, the speech decoder can be trained on training data the captures the relationship between neural signals and para-linguistic characteristics such that the speech output by the speech decoder better reflects the subject's intended speech patterns.

With reference to FIG. 2C, illustrated is an alternate technique for aligning the biosignals of a subject attempting to speak a target speech sequence and synthetic speech representative of the target speech signal. In the illustrated scenario, the subject may be dysarthric and still able to produce audible, but unintelligible, speech. Accordingly, in the illustrated scenario, the biosignal may be an audio signal 215c representative of the subject attempting to speak the target speech sequence. It should be appreciated that EMG, AMG, MMG, breathing, audio, image, and/or ultrasound sensors may be additionally or alternatively utilized to improve the segmentation of the biosignals.

The systems disclosed herein may then align a synthetic speech signal 225 (such as the synthetic speech signal 125) to the audio signal 215c. As illustrated, the alignment process may perform time-warping operations such that the characteristic waveform of the synthetic speech signal is mapped into the envelop of the corresponding portion of the audio signal 215c. In some embodiments, the time-warping technique may include one or more of the approaches described with respect to FIGS. 2A-B. In some embodiments, the alignment process includes aligning the para-linguistic characteristics of the audio signal 215c when generating the target speech signal 227c in a similar manner as described with respect to FIGS. 2A-B. After the mapping operation is complete, the resulting target speech signal 227c generally reflects a speech pattern if the subject were to be able to actually speak the target speech sequence.

Additionally, because the biosignals 215 and the target speech signals 227 are aligned in time, the underlying relationships between the biosignal signal 215 and target speech signals 227 can be used to train a speech decoder (such as the speech decoder 130) to operate in a continuous manner. Similarly, the alignment of the biosignals 215 and the target speech signals 227 enable the speech decoder to learn the causal relationship between the biosignal inputs and the target speech outputs. Thus, unlike conventional speech decoders, a speech decoder trained using the aligned training data in accordance with the techniques disclosed herein are able to train a causal speech decoder that operates in a continuous manner.

Accordingly, unlike conventional speech decoders, a speech decoder trained using the instant techniques can restore real-time “speech” capabilities to anarthric subjects.

Returning to FIG. 1A, the speech decoder 130 is trained using the neural signals 115 (and any other biosignals) as an input signal and an aligned version of the synthetic speech signal 125 (a “target speech signal,” such as the target speech signals 227a, 227b, 227c) as the ground truth. In some embodiments, the speech decoder 130 is a Transformer model, such as the Transformer model disclosed in Vaswani, A.; Shazeer, N.; Parmar, N.; Uszkoreit, J.; Jones, L.; Gomez, A. N.; Kaiser, Ł.; and Polosukhin, I. 2017. Attention is all you need. In Advances in Neural Information Processing Systems, 5998-6008.

In other embodiments, the speech decoder may be a recurrent neural network (RNN) model. In one example, the RNN may include four layers with 512 gated recurrent units (GRUs) each, 20% dropout between layers, 50% recurrent dropout within each layer, with tanh activation and sigmoid recurrent activation. In this example, the RNN may then include a sequential weighted attention layer and a dense layer to predict the output vector. It should be appreciated that this example is one example RNN structure suitable for use in a speech decoder, and other RNNs having a different structure may be used in other embodiments. For example, some alternate RNNs may be enhanced with a Transformer model (e.g., an RNN-T model). While the foregoing describes RNN architectures, the techniques described herein may be implemented using any neural network architecture suitable for time-series modeling may be used alone or in combination with one another. Regardless of the particular neural network architecture, the hyperparameters of the model structure may be chosen and/or mathematically derived based upon the based upon the particular model architecture, the particular biosignals input into the model, and/or characteristics of the subject.

In some embodiments, a supervised learning approach is used to train the speech decoder 130. To prepare the training data, 100 ms of neural data (t-9 to t bins) may be extracted from the neural signal 115 as input. The extracted neural data may then be used to compute the corresponding speech features at time t as the target output of the speech decoder for input into an encoder, such as the LPCNet encoder or other type of encoder optimized for human speech (e.g., a “vocoder”). The LPCNet encoder converts an input audio waveform to low-dimensional speech features characterized by 18 cepstral coefficients-pitch period, pitch strength and 16 linear predictive coding (LPC) coefficients. To reduce dimensionality of the output further for ease of training the speech decoder 130, only the first 8 cepstral coefficients, pitch period, and pitch strength may be used resulting in a 10-D speech representation from which intelligible speech can still be reconstructed. It should be appreciated that while the LPCNet encoder is described above, other vocoder may be utilized to encode the input audio waveforms. Additionally, while the foregoing describes a vocoder approach to generating synthesized speech, in other embodiments, an encoder may be a kinematic and/or kinetic model that generates speech using a vocal tract model. Thus, in these alternate embodiments, the output feature space may be adapted to the particular kinematic and/or kinetic model used to encode speech.

In one example training process, the speech decoder 130 is trained on minibatches of 1024 samples, each with 10 neural data bins (100 ms) and the corresponding output speech feature vector (the corresponding bins of the target speech signal) over 15 epochs that use the entire training set to train the speech decoder 130. In one example, the learning rate is set to 0.001 and reduced exponentially after each epoch. In this example, the gradients may be updated after each minibatch, for example, using an Adam optimizer and Huber loss with 5=1.3 for regression. See D. P. Kingma and J. Ba, “Adam: A method for stochastic optimization,” arXiv preprint arXiv: 1412.6980, 2014 and P. J. Huber, “Robust Estimation of a Location Parameter,” The Annals of Mathematical Statistics, vol. 35, no. 1, pp. 73-101, 3 1964. Updating the gradients in this manner combines advantages of both L1 and L2 loss and is less sensitive to outliers.

In some embodiments, to measure performance through the training process, the output synthesized speech (generated by a vocoder and/or a vocal tract model from speech features estimated by the neural decoder) may be compared with ground truth data (e.g., the target speech sequence generated using the above-described techniques). In particular, the comparison may determine the average Pearson correlation coefficient across 40 Mel frequencies between the synthesized speech spectrum and the true speech spectrum.

FIG. 1B depicts an example environment 150 for applying the trained speech decoder 130 to produced synthesized speech 177. As illustrated, FIG. 1B includes a BCI 155 coupled with the brain 112 of the subject 110. The BCI 155 may be coupled to the subject 110 in a similar manner as the BCI 105. Additionally, other sensors described with respect to FIG. 1A may also be coupled with the subject 110.

As illustrated, the subject 110 may generate a speech intention (“I am feeling fine”), for example, via attempted speech. The neural signals 165 may reflect the neural activity as the subject 110 generates the speech intention. Similarly, any other sensors may also generate a corresponding biosignal reflecting the speech intention.

The neural signals 165 (and any other biosignals) are then input into the speech decoder 130 trained in the manner described with respect to FIG. 1A. Accordingly, the speech decoder 130 may output a speech signal encoded into a format associated with a speech synthesizer (not depicted). As described herein, the speech synthesizer may be a vocoder, a kinetic model, a kinematic model, or other type of speech synthesis model configured to produce synthesized speech 167 representative of the speech intention of the subject 110. The synthesized speech 167 may then output to an audio output device (e.g., a speaker) to produce an audio output 177 and/or transcribed and displayed on a display device as a text output 178.

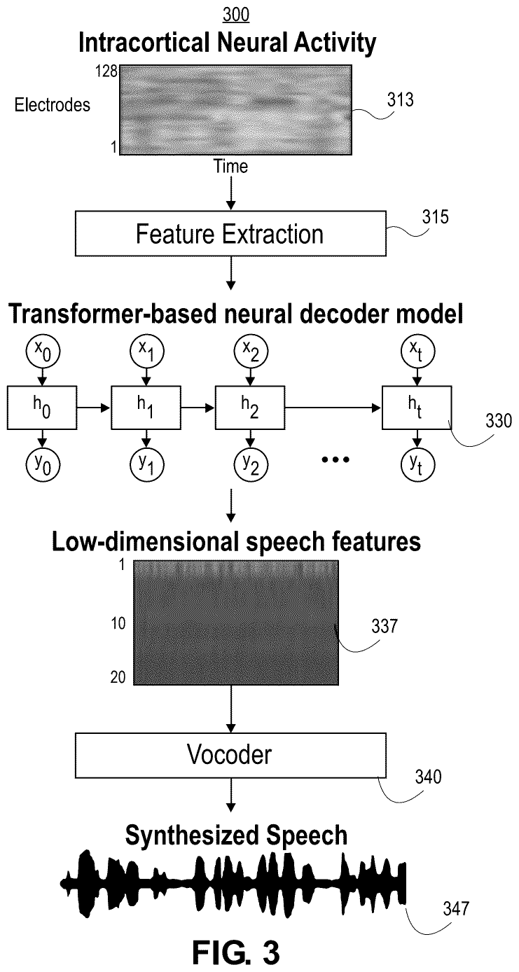

Turning to FIG. 3, illustrated is an example decoder architecture 300 for speech synthesis from neural activity 313 in accordance with one embodiment of the disclosed techniques. The architecture 300 may reflect the process flow for generating a synthesized speech inference using a speech decoder model 330, such as a speech decoder model trained using the techniques described with respect to FIGS. 1A-2C.

In the example architecture 300, the first layer is the detection of neural activity 313.

For example, the neural activity 313 may be the raw data from each of the electrodes included in a BCI (such as the BCI 105). As illustrated, the second layer includes a feature extraction layer 315 via which the neural activity is analyzed to produce a neural signal. Generally, the feature extraction layer 315 applies the same techniques to generate the one or more biosignals on which the speech decoder 330 was trained to generate the neural signal. For example, the techniques used to generate the neural signals 215 may be applied to extract the features of the neural activity 313 that form the neural signal.

As described herein, in some alternate embodiments, the neural signal may be supplemented or replaced with other biosignals. Accordingly, if the training process for the speech decoder 330 included one or more of these other biosignals, the feature extraction layer 315 may also process the corresponding sensor data to produce the supplementary one or more biosignals.

It should be appreciated that feature extraction 315 may be configured to continuously generate one or more biosignals from the subject. Accordingly, the feature extraction layer 315 may include a buffer that stores a most recent portion of the extracted biosignals. For example, the feature extraction layer 315 may include binning the biosignals into segments of a predetermined length (e.g., 1 ms, 2 ms, 3 ms, 5 ms, 10 ms, 20 ms, 50 ms, 100 ms, 200 ms, etc.) generally corresponding to the update rate of the speech decoder 330. The buffer may then store at least a predetermined number of binned biosignals (e.g., 100 ms of bins, 200 ms of bins, 400 ms of bins, 600 ms of bins, 1 second of bins, etc.) generally associated with a window size for the speech decoder 330. It should be appreciated that the window size may vary depending on the individual subject and/or the signals derived therefrom. For example, the window size may reflect the length of a causal biosignal window. As another example, the window size may change based on the model architecture to minimize the model training time or computational resources.

The one or more biosignals generated by the feature extraction layer 315 are then input into the trained speech decoder 330 to causally produce a low-dimensional feature vector 337 that encodes an output speech waveform. More particularly, a set of the binned biosignals (e.g., an input window) may be input into the trained speech decoder 330 to produce a frame of encoded speech. Depending on the embodiment, the frame of encoded speech (e.g., an update rate) may encode, for example, 1 ms of speech data, 2 ms of speech data, 3 ms of speech data, 5 ms of speech data, 10 ms of speech data, 20 ms of speech data, 50 ms of speech data, 100 ms of speech data, or 200 ms of speech data.

In the illustrated example, the feature vector 337 for the frame of encoded speech produced by the speech decoder 337 is input into a vocoder 340 to produce the synthesized speech 347. The synthesized speech may then be output via an audio output device (e.g., a speaker) in real-time as the frames of speech are encoded. Accordingly, the features of the feature vector 337 may correspond to particular input channels of the selected vocoder 340.

The particular number of features included in the feature vector 337 may vary depending on the vocoder 340 and/or a decision to omit input features of a vocoder from the training process to improve the speed of training. As described above, the LPCNet vocoder may include utilize a 20-dimension to 36-dimension input vector.

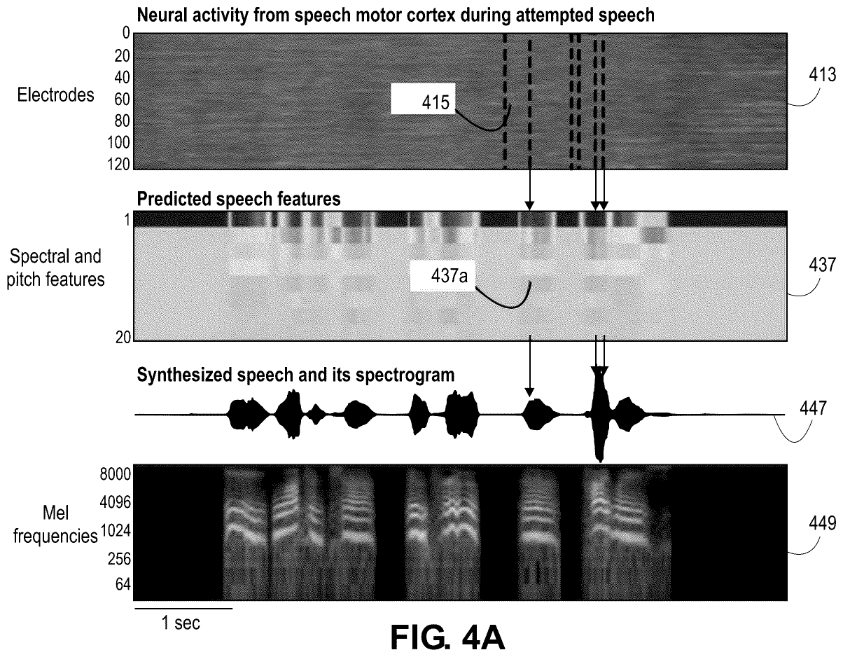

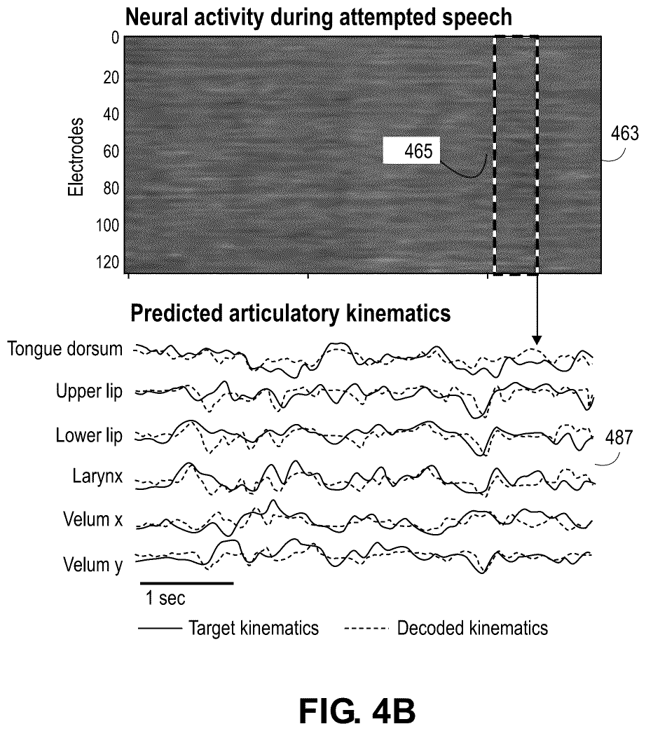

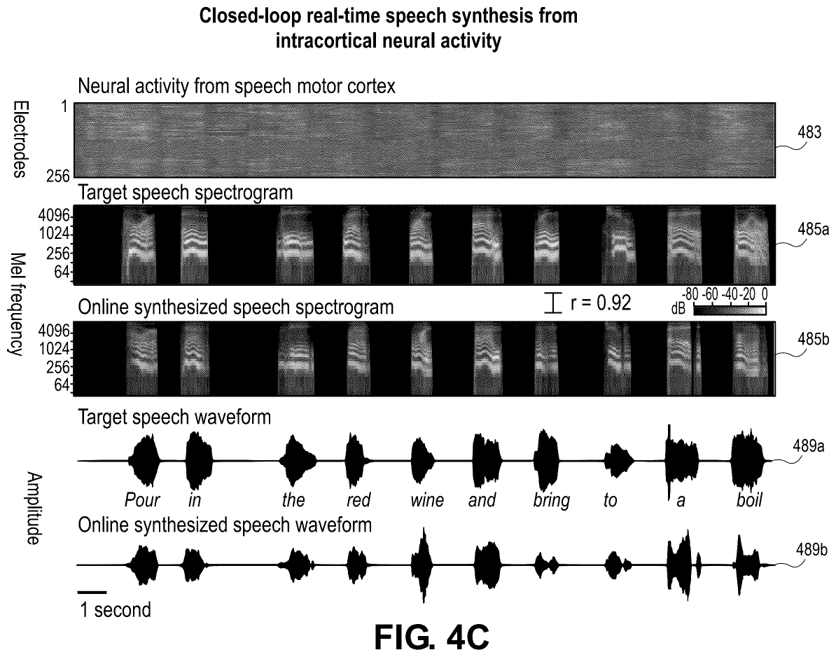

With simultaneous reference to FIG. 4A, illustrated is an example diagram showing the encoding of neural signals 413 (such as the neural signals 313) into its component feature vectors 437 to produce synthesized speech 447. As illustrated, bins of neural signals 415a representative of 100 ms of neural activity 413 are input into a speech decoder (such as the speech decoder 330) to a feature vector 437a in a 20-dimensional feature space. The feature vector 437a is then input into a vocoder, such as the vocoder 340, to produce the synthesized speech signal 447. For reference, the corresponding Mel frequencies 449 of the synthesized speech signal 447 is also depicted.

It should be appreciated that while the while architecture 300 includes a vocoder 340, in other embodiments, the speech decoder 330 may instead be configured to produce one or more kinematic and/or kinetic signal outputs instead of the low-dimensional feature vector 337. With simultaneous reference to FIG. 4B, illustrated is an example diagram showing the encoding of neural signals 463 (such as the neural signals 313) into component kinematic and/or kinetic signals to produce synthesized speech.

Similar to FIG. 4A, bins of neural signals 465a representative of 100 ms of neural activity 463 are input into a speech decoder. However, unlike the speech decoder 330, the speech decoder is instead trained to output component kinematic signals 487 representative of the movement of speech articulators. For example, as illustrated, the kinematic signals may include a tongue dorsum signal, an upper lip signal, a lower lip signal, a larynx signal, a velum x signal, and a velum y signal. The kinematic signals 487 are then input into to a vocal tract model configured to convert the kinematic movement encoded by the kinematic signal 487 into a synthesized speech signal. In other embodiments, the speech decoder is configured to output component kinetic signals.

FIG. 4C depicts an example diagram depicting experimental results of a speech decoder trained in accordance with the disclosed techniques (such as the speech decoders 130, 330) producing accurate synthesized speech in real-time (e.g., “online” speech or synthesized speech produced within 10 ms, 5 ms, etc. of detecting the representative neural signal 483). In the illustrated example, the neural signal 483 is captured from a subject diagnosed with amyotrophic lateral sclerosis (ALS) and attempt to speak the phrase “Pour in the red wine and bring to a boil.” The synthesized speech output by a vocoder (such as the synthesized speech 167, 347) is compared to a target speech signal (such as the target speech signals 227).

As part of the comparison, FIG. 4C depicts the spectrogram across the 40 Mel frequencies for the target speech signal (spectrogram 485a) and the synthesized speech signal (spectrogram 485b). The Pearson correlation between spectrograms 485a and 485b is 0.92 which indicates a high degree of similarity. Additionally, the FIG. 4C. depicts the speech waveforms 489a and 489b for the target speech signal and the synthesized speech signal, respectively. As illustrated, the segment timing and envelop shape are also similar, indicating that the synthesized speech well-represents the target speech signal.

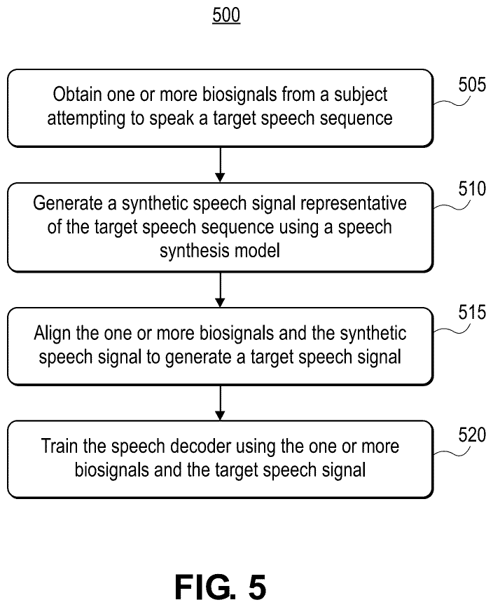

Turning to FIG. 5, illustrated is a flowchart of an example method 500 for training a speech decoder (such as the speech decoders 130, 330), for example, to restore speech to an anarthric or dysarthric subject. The method may be performed by one or more processors of any suitable computing system. In some embodiments, the computing system may be a cloud computing system in which the one or more processors are distributed among a plurality of hardware units. In some other embodiments, the computing system may include a local system that includes central processing units (CPUs) and/or graphical processing units (GPUs).

The method 500 begins at block 505 when the processors obtain one or more biosignals from a subject (such as the subject 110) attempting to speak a target speech sequence (such as the target speech sequence 107). In some embodiments, the one or more biosignals (such as the biosignals 115, 215a, 215b, 215c) are derived from data obtained from a sensor, wherein the sensor is selected from the group consisting of an intracortical electrode array, an electrocorticography (EcoG) sensor, a stereoelectroencephalography (sEEG) sensor, a surface electroencephalography (EEG) sensor, a functional near-infrared spectroscopy (fNIRS) sensor, a magnetoencephalography (MEG) sensor, an electromyography (EMG) sensor, an acoustic myography (AMG) sensor, a magnetomyography (MMG) sensor, an ultrasound sensor, a light detection and ranging (LiDAR) sensor, an infrared sensor, a video sensor, an audio sensor, a vibration sensor, a motion sensor, an inertial measurement unit (IMU), a piezoelectric sensor, a breathing sensor, another intracortical device capable of recording individual neurons, and any combination thereof. For sensors that are coupled with the subject's brain, the sensor may be coupled to a motor cortex of the subject (e.g., the ventral motor cortex or the dorsal motor complex), an inferior frontal gyrus, a superior temporal gyrus, basal ganglia, a cerebellum, a brainstem, a thalamus, or a subthalamic nucleus of the subject, or another brain location associated with speech neural activity.

At block 510, the processors generate a synthetic speech signal (such as the synthetic speech signals 125, 225) representative of the target speech sequence using a speech synthesis model (such as the TTS model 120). In some embodiments, the processors may generate the synthetic speech by generating a kinematic and/or kinetic model of articulators of a person speaking the target speech sequence and inputting an output of the kinematic and/or kinetic model into the speech synthesis model to produce the synthetic speech signal. In other embodiments, the processors may generate the synthetic speech by obtaining historical speech data associated with the subject and training the speech synthesis model using the historical speech data. In still other embodiments, the processors may generate the synthetic speech by obtaining one or more voice characteristics of the subject and selecting the speech synthesis model based upon the one or more voice characteristics. In these embodiments, the one or more voice characteristics may be selected form the group consisting of gender, age, country of origin, accent, and any combination thereof.

At block 515, the processors align the one or more biosignals and the synthetic speech signal to generate a target speech signal (such as the target speech signals 227a, 227b, 227c). More particularly, the processors may analyze the one or more biosignals to identify samples corresponding to individual words, syllables, phonemes, and/or other sounds of the target speech sequence and map the samples to corresponding segments of the synthetic speech signal. In embodiments where the one or more biosignals include an audio signal (such as the biosignal 215b) of the subject attempting to speak the target speech sequence, the processors may identify the sample by identifying breaks included in the audio signal. In embodiments where the one or more biosignals include a neural signal (such as the biosignal 215a) of the subject attempting to speak the target speech sequence, the processors may identify the sample by identifying dynamics of neural activity in the one or more neural signals.

In some embodiments, the processors may identify the samples by (i) inputting the one or more biosignals into a second speech decoder (such as a previously-trained version of the speech decoder or another speech decoder altogether) to produce a second encoded speech signal; (ii) analyzing the second encoded speech signal to identify sample timing; and (iii) applying the identified sample timing to the one or more biosignals. In some additional embodiments, the processors may identify the samples by inputting the one or more biosignals into a text prediction model (such as a text prediction layer included in the speech decoder 330) configured to output a transcript of speech represented by the one or more biosignals and identifying the one or more biosignals based on the transcript. In further examples, the processors may identify the samples by inputting the one or more biosignals into a time-series model (such as a Hidden Markov Model or a Connectionist Temporal Classification model) configured to predict sample timings when each sample occurred and applying the predicted sample timings to the one or more biosignals.

In either embodiment, the processors may apply a time-warping function to the samples to align the durations of the samples with the corresponding segment of the synthetic speech signal. Additionally, the processors may apply a para-linguistics matching function to the corresponding segment of the synthetic speech signal to match para-linguistic characteristics of the subject's speech (e.g., pitch, intonation, prosody, and volume) such that paralinguistic characteristics of the attempted speech represented by the one or more biosignals are exhibited by the synthetic speech signal.

At block 520, the processors train the speech decoder using the one or more biosignals and the target speech signal. The speech decoder may be a Transformer model, a recurrent neural network (RNN), and/or any other neural network architecture capable of modeling time-series data configured to output an encoded speech signal. In some embodiments, the encoded speech signal is encoded in a format configured to be input into a vocoder (such as the vocoder 340) that generates synthesized speech. In other embodiments, the encoded speech signal is encoded in a format configured to be input into a vocal tract model for synthesizing speech from articulatory kinematics and/or kinetics.

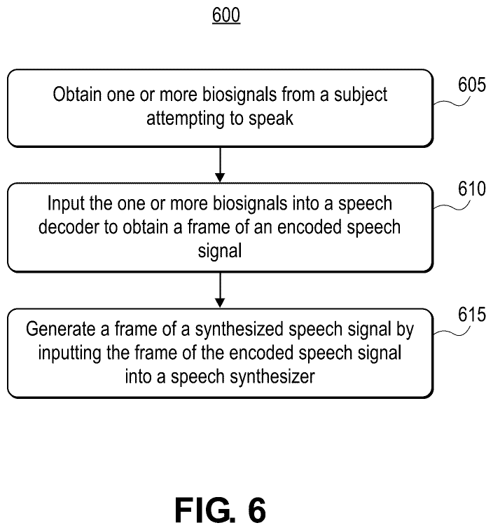

Turning to FIG. 6, illustrated is a flowchart of an example method 600 for generating synthesized speech, for example, to restore speech to an anarthric or dysarthric subject. The method may be performed by one or more processors of any suitable computing system. In some embodiments, the computing system may be a cloud computing system in which the one or more processors are distributed among a plurality of hardware units. In some other embodiments, the computing system may include a local system that includes central processing units (CPUs) and/or graphical processing units (GPUs). It should be appreciated that the processors configured to perform the method 600 may be different than the processors configured to perform the method 500.

The method 600 begins at block 605 when the processors receive one or more biosignals (such as the biosignals 165, 415, 465, 483) recorded from a subject attempting to speak. In some embodiments, the one or more biosignals are derived from data obtained from a sensor, wherein the sensor is selected form the group consisting of an intracortical electrode array, an electrocorticography (ECOG) sensor, a stereoelectroencephalography (sEEG) sensor, a surface electroencephalography (EEG) sensor, a functional near-infrared spectroscopy (fNIRS) sensor, a magnetoencephalography (MEG) sensor, an electromyography (EMG) sensor, an acoustic myography (AMG) sensor, a magnetomyography (MMG) sensor, an ultrasound sensor, a light detection and ranging (LiDAR) sensor, an infrared sensor, a video sensor, an audio sensor, a vibration sensor, a motion sensor, an inertial measurement unit (IMU), a piezoelectric sensor, a breathing sensor, another intracortical device capable of recording individual neurons, and any combination thereof. For sensors that are coupled with the subject's brain, the sensor may be coupled to a motor cortex of the subject (e.g., the ventral motor cortex or the dorsal motor complex), an inferior frontal gyrus, a superior temporal gyrus, basal ganglia, a cerebellum, a brainstem, a thalamus, or a subthalamic nucleus of the subject, or another brain location associated with speech neural activity.

In some embodiments, the processors may store the one or more biosignals in a buffer. For example, the processors may generate 10 ms bins of the one or more biosignals.

At block 610, the processors input the one or more biosignals into a speech decoder (such as the speech decoders 130, 330) to obtain a frame (such as frames 437a, 487a) of an encoded speech signal (such as signals 337, 437, 487). The speech decoder may be trained in accordance with the method 500. Accordingly, the speech decoder may be trained using training data that includes aligned historic biosignals associated with the subject attempting to speak a target speech sequence and synthetic speech for the target speech sequence. The speech decoder may include a Transformer model, a recurrent neural network (RNN), and/or any other neural network architecture capable of modeling time-series data.

In embodiments that include the buffer, the processors may obtain a segment of the one or more biosignals from the buffer and input the segment of the one or more biosignals into the speech decoder. In some embodiments, the segment is representative of at least 100 ms of the one or more biosignals. In other embodiments, the segment is representative of at least 200 ms of the one or more biosignals. In still other embodiments, the segment is representative of at least 400 ms of the one or more biosignals. In even further embodiments, the segment is representative of at least 600 ms of the one or more biosignals.

At block 615, the processors generate a frame of a synthesized speech signal (such as signal 167, 347, 447) by inputting the frame of the encoded speech signal into a speech synthesizer (such as the vocoder 340). The speech decoder may be configured to generate the frame of the synthesized speech signal within about 10 milliseconds of obtaining the of one or more biosignals. This time generally corresponds to an update rate for the speech decoder. In some embodiments in which the update rate is faster (e.g., when improved processing power enables faster computation of the encoded speech signal), the speech decoder may be configured to generate the frame of the synthesized speech signal within about 5 milliseconds, 3 milliseconds, 2 milliseconds, or one millisecond of obtaining the of one or more biosignals. In some embodiments in which the update rate is slower (e.g., when the speech decoder is implemented using less expensive computing systems), the speech decoder may be configured to generate the frame of the synthesized speech signal within about 20 milliseconds, 50 milliseconds, 100 milliseconds, 150 milliseconds, or 200 milliseconds of obtaining the of one or more biosignals.

In some embodiments, the frame of the encoded speech signal is encoded in a format configured to be input into a vocoder that generates the frame of synthesized speech (such as shown in FIG. 4A). In other embodiments, the encoded speech signal is encoded in a format configured to be input into a vocal tract model for synthesizing speech from articulatory kinematics and/or kinetics (such as shown in FIG. 4B). Regardless, in some embodiments, in response to generating the frame of the synthesized speech signal, the synthesized speech is output via an audio output device (e.g., a speaker) and/or transcribed and presented via a display device.

### EXAMPLE EMBODIMENTS

Embodiment 1. A computer-implemented method for training a speech decoder, the method comprising: obtaining, via one or more processors, one or more biosignals from a subject attempting to speak a target speech sequence; generating, via the one or more processors, a synthetic speech signal representative of the target speech sequence using a speech synthesis model; aligning, via the one or more processors, the one or more biosignals and the synthetic speech signal to generate a target speech signal; and training, via the one or more processors, the speech decoder using the one or more biosignals and the target speech signal, wherein the speech decoder is configured to output an encoded speech signal.

Embodiment 2. The computer-implemented method of Embodiment 1, wherein the one or more biosignals are derived from data obtained from a sensor, wherein the sensor is selected from the group consisting of an intracortical electrode array, an electrocorticography (EcoG) sensor, a stereoelectroencephalography (sEEG) sensor, a surface electroencephalography (EEG) sensor, a functional near-infrared spectroscopy (fNIRS) sensor, a magnetoencephalography (MEG) sensor, an electromyography (EMG) sensor, an acoustic myography (AMG) sensor, a magnetomyography (MMG) sensor, an ultrasound sensor, a light detection and ranging (LiDAR) sensor, an infrared sensor, a video sensor, an audio sensor, a vibration sensor, a motion sensor, an inertial measurement unit (IMU), a piezoelectric sensor, a breathing sensor, another intracortical device capable of recording individual neurons, and any combination thereof.

Embodiment 3. The computer-implemented method of Embodiment 2, wherein the sensor is coupled with a motor cortex of the subject.

Embodiment 4. The computer-implemented method of Embodiment 2, wherein the sensor is coupled with an inferior frontal gyrus, a superior temporal gyrus, basal ganglia, a cerebellum, a brainstem, a thalamus, or a subthalamic nucleus of the subject.

Embodiment 5. The computer-implemented method of any one of Embodiments 1 to 4, wherein generating the synthetic speech comprises: generating, via the one or more processors, a kinematic and/or kinetic model of articulators of a person speaking the target speech sequence; and inputting, via the one or more processors, an output of the kinematic and/or kinetic model into the speech synthesis model to produce the synthetic speech signal.

Embodiment 6. The computer-implemented method of any one of Embodiments 1 to 5, wherein generating the synthetic speech comprises: obtaining, via the one or more processors, historical speech data associated with the subject; and training, via the one or more processors, the speech synthesis model using the historical speech data.

Embodiment 7. The computer-implemented method of any one of Embodiments 1 to 5, wherein generating the synthetic speech comprises: obtaining, via the one or more processors, one or more voice characteristics of the subject; and selecting, via the one or more processors, the speech synthesis model based upon the one or more voice characteristics.

Embodiment 8. The computer-implemented method of Embodiment 7, wherein the one or more voice characteristics are selected form the group consisting of gender, age, country of origin, accent, speech style, and any combination thereof.

Embodiment 9. The computer-implemented method of any one of Embodiments 1 to 8, wherein aligning the one or more biosignals and the synthetic speech signal comprises: analyzing, via the one or more processors, the one or more biosignals and the synthetic speech signal to identify corresponding samples representative of individual words, syllables, phonemes, and/or other sounds of the target speech sequence; and mapping, via the one or more processors, the samples of the synthetic speech signal to corresponding sample of the one or more biosignals.

Embodiment 10. The computer-implemented method of Embodiment 9, wherein: the one or more biosignals include an audio signal of the subject attempting to speak the target speech sequence; and identifying the samples comprises segmenting, by the one or more processors, the audio signal based on breaks included in the audio signal.

Embodiment 11. The computer-implemented method of Embodiment 9, wherein: the one or more biosignals include one or more neural signals of the subject attempting to speak the target speech sequence; and identifying the samples comprises identifying, by the one or more processors, dynamics of neural activity in the one or more neural signals.

Embodiment 12. The computer-implemented method of Embodiment 9, wherein identifying the samples comprises: inputting, via the one or more processors, the one or more biosignals into a second speech decoder to produce a second encoded speech signal; analyzing, via the one or more processors, the second encoded speech signal to identify sample timing; and applying, via the one or more processors, the identified sample timing to algin the synthetic speech signal with the one or more biosignals.

Embodiment 13. The computer-implemented method of Embodiment 9, wherein identifying the samples comprises: inputting, via the one or more processors, the one or more biosignals into a text prediction model configured to output a transcript and corresponding timings of speech represented by the one or more biosignals; and identifying, via the one or more processors, the one or more biosignals based on the transcript and the corresponding timings.

Embodiment 14. The computer-implemented method of Embodiment 9, wherein identifying the samples comprises: inputting, via the one or more processors, the one or more biosignals into a time-series model configured to predict sample timings when each sample occurred; and applying, via the one or more processors, the predicted sample timings to align the synthetic speech signal with the one or more biosignals.

Embodiment 15. The computer-implemented method of any one of Embodiments 9 to 14, wherein mapping the samples comprises: applying, via the one or more processors, a time-warping function to the samples to align durations of the samples with the corresponding segment of the one or more biosignals.

Embodiment 16. The computer-implemented method of any one of Embodiments 9 to 15, wherein mapping the samples comprises: applying, via the one or more processors, a para-linguistics matching function to the corresponding segment of the synthetic speech signal such that paralinguistic characteristics of the attempted speech represented by the one or more biosignals are exhibited by the synthetic speech signal.

Embodiment 17. The computer-implemented method of any one of Embodiments 1 to 16, wherein the speech decoder includes a Transformer model.

Embodiment 18. The computer-implemented method of any one of Embodiments 1 to 17, wherein the speech decoder includes a recurrent neural network (RNN).

Embodiment 19. The computer-implemented method of any one of Embodiments 1 to 18, wherein the encoded speech signal is encoded in a format configured to be input into a vocoder that generates synthesized speech.

Embodiment 20. The computer-implemented method of any one of Embodiments 1 to 19, wherein the encoded speech signal is encoded in a format configured to be input into a vocal tract model for synthesizing speech from articulatory kinematics and/or kinetics.

Embodiment 21. The computer-implemented method of any one of Embodiments 1 to 20 wherein the subject is anarthric or dysarthric.

Embodiment 22. A system comprising: one or more processors; and a non-transitory memory storing processor-executable instructions that, when executed by the one or more processors, cause the system to perform the method of any one of Embodiments 1 to 21.

Embodiment 23. A non-transitory computer-readable storage medium storing processor-executable instructions that, when executed by one or more processors, cause the one or more processors to perform the method of any one of Embodiments 1 to 21.

Embodiment 24. A computer-implemented method for generating synthesized speech, the method comprising: obtaining, via one or more processors, one or more biosignals from a subject attempting to speak; inputting, via the one or more processors, the one or more biosignals into a speech decoder to obtain a frame of an encoded speech signal, wherein the speech decoder is trained using training data that includes aligned historic biosignals associated with the subject attempting to speak a target speech sequence and synthetic speech for the target speech sequence; and generating, via the one or more processors, a frame of a synthesized speech signal by inputting the frame of the encoded speech signal into a speech synthesizer, wherein the speech decoder is configured to generate the frame of synthesized speech signal within about 10 milliseconds of obtaining the one or more biosignals.

Embodiment 25. The computer-implemented method of Embodiment 24, wherein the speech decoder is configured to generate the frame of the synthesized speech signal within about 5 milliseconds of obtaining the one or more biosignals.

Embodiment 26. The computer-implemented method of Embodiment 24 or 25, further comprising: storing, via the one or more processors, the one or more biosignals in a buffer.

Embodiment 27. The computer-implemented method of Embodiment 26, wherein inputting the one or more biosignals into the speech decoder further comprises: obtaining, via the one or more processors, a segment of the one or more biosignals from the buffer; and inputting, via the one or more processors, the segment of the one or more biosignals into the speech decoder.

Embodiment 28. The computer-implemented method of Embodiment 27, wherein the segment is representative of at least 100 milliseconds of the one or more biosignals.

Embodiment 29. The computer-implemented method of Embodiment 28, wherein the segment is representative of at least 200 milliseconds of the one or more biosignals.

Embodiment 30. The computer-implemented method of any one of Embodiments 24 to 29, wherein the segment is representative of at least 600 milliseconds of the one or more biosignals.

Embodiment 31. The computer-implemented method of any one of Embodiments 24 to 30, wherein the one or more biosignals are derived from data obtained from a sensor, wherein the sensor is selected from the group consisting of an intracortical electrode array, an electrocorticography (ECOG) sensor, a stereoelectroencephalography (sEEG) sensor, a surface electroencephalography (EEG) sensor, a functional near-infrared spectroscopy (fNIRS) sensor, a magnetoencephalography (MEG) sensor, an electromyography (EMG) sensor, an acoustic myography (AMG) sensor, an ultrasound sensor, a magnetomyography (MMG) sensor, a light detection and ranging (LiDAR) sensor, an infrared sensor, a video sensor, an audio sensor, a vibration sensor, a motion sensor, an inertial measurement unit (IMU), a piezoelectric sensor, a breathing sensor, another intracortical device capable of recording individual neurons, and any combination thereof.

Embodiment 32. The computer-implemented method of Embodiment 31, wherein the sensor is coupled with a motor cortex of the subject.

Embodiment 33. The computer-implemented method of Embodiment 31, wherein the sensor is coupled with an inferior frontal gyrus, a superior temporal gyrus, basal ganglia, a cerebellum, a brainstem, a thalamus, or a subthalamic nucleus of the subject.

Embodiment 34. The computer-implemented method of any one of Embodiments 24 to 33, wherein the speech decoder includes a Transformer model.

Embodiment 35. The computer-implemented method of any one of Embodiments 24 to 34, wherein the speech decoder includes a recurrent neural network (RNN).

Embodiment 36. The computer-implemented method of any one of Embodiments 24 to 35, wherein the frame of the encoded speech signal is encoded in a format configured to be input into a vocoder that generates synthesized speech.

Embodiment 37. The computer-implemented method of any one of Embodiments 24 to 35, wherein the encoded speech signal is encoded in a format configured to be input into a vocal tract model for synthesizing speech from articulatory kinematics and/or kinetics.

Embodiment 38. The computer-implemented method of any one of Embodiments 24 to 37, wherein the subject is anarthric or dysarthric.

Embodiment 39. The computer-implemented method of any one of Embodiments 24-28, further comprising: outputting, via an audio output device, the frame of the synthesized speech signal.

Embodiment 40. A system comprising: one or more processors; and a non-transitory memory storing processor-executable instructions that, when executed by the one or more processors, cause the system to perform the method of any one of Embodiments 24 to 39.

Embodiment 41. A non-transitory computer-readable storage medium storing processor-executable instructions that, when executed by one or more processors, cause the one or more processors to perform the method of any one of claims 24 to 39.

### ADDITIONAL CONSIDERATIONS

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated.

These and other variations, modifications, additions, and improvements fall within the scope of the subject matter herein.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of “a” or “an” is employed to describe elements and components of the embodiments herein. This is done merely for convenience and to give a general sense of the invention. This description should be read to include one or at least one and the singular also includes the plural unless it is obvious that it is meant otherwise. The terms “substantially”, “essentially”, “approximately”, “about” or any other version thereof, are defined as being close to as understood by one of ordinary skill in the art, and in one non-limiting example the term is defined to be within 10%, in another example within 5%, in another example within 1% and in another example within 0.5%.

Upon reading this disclosure, those of skill in the art will appreciate still additional alternative structural and functional designs for identifying and grouping likely textual near-duplicates through the principles disclosed herein. Thus, while particular embodiments and applications have been illustrated and described, it is to be understood that the disclosed embodiments are not limited to the precise construction and components disclosed herein.

Various modifications, changes and variations, which will be apparent to those skilled in the art, may be made in the arrangement, operation and details of the method and apparatus disclosed herein without departing from the spirit and scope defined in the appended claims.

What is claimed:

1. A computer-implemented method for training a speech decoder, the method comprising:
obtaining, via one or more processors, one or more biosignals from a subject attempting to speak a target speech sequence;
generating, via the one or more processors, a synthetic speech signal representative of the target speech sequence using a speech synthesis model;
aligning, via the one or more processors, the one or more biosignals and the synthetic speech signal to generate a target speech signal; and
training, via the one or more processors, the speech decoder using the one or more biosignals and the target speech signal, wherein the speech decoder is configured to output an encoded speech signal.

2. The computer-implemented method of claim 1, wherein the one or more biosignals are derived from data obtained from a sensor, wherein the sensor is selected from the group consisting of an intracortical electrode array, an electrocorticography (EcoG) sensor, a stereoelectroencephalography (sEEG) sensor, a surface electroencephalography (EEG) sensor, a functional near-infrared spectroscopy (fNIRS) sensor, a magnetoencephalography (MEG) sensor, an electromyography (EMG) sensor, an acoustic myography (AMG) sensor, a magnetomyography (MMG) sensor, an ultrasound sensor, a light detection and ranging (LiDAR) sensor, an infrared sensor, a video sensor, an audio sensor, a vibration sensor, a motion sensor, an inertial measurement unit (IMU), a piezoelectric sensor, a breathing sensor, another intracortical device capable of recording individual neurons, and any combination thereof.

3. The computer-implemented method of claim 2, wherein the sensor is coupled with a motor cortex of the subject.

4. The computer-implemented method of claim 2, wherein the sensor is coupled with an inferior frontal gyrus, a superior temporal gyrus, basal ganglia, a cerebellum, a brainstem, a thalamus, or a subthalamic nucleus of the subject.

5. The computer-implemented method of any one of claims 1 to 4, wherein generating the synthetic speech comprises:
generating, via the one or more processors, a kinematic and/or kinetic model of articulators of a person speaking the target speech sequence; and
inputting, via the one or more processors, an output of the kinematic and/or kinetic model into the speech synthesis model to produce the synthetic speech signal.

6. The computer-implemented method of any one of claims 1 to 5, wherein generating the synthetic speech comprises:
obtaining, via the one or more processors, historical speech data associated with the subject; and
training, via the one or more processors, the speech synthesis model using the historical speech data.

7. The computer-implemented method of any one of claims 1 to 5, wherein generating the synthetic speech comprises:
obtaining, via the one or more processors, one or more voice characteristics of the subject; and
selecting, via the one or more processors, the speech synthesis model based upon the one or more voice characteristics.

8. The computer-implemented method of claim 7, wherein the one or more voice characteristics are selected form the group consisting of gender, age, country of origin, accent, speech style, and any combination thereof.

9. The computer-implemented method of any one of claims 1 to 8, wherein aligning the one or more biosignals and the synthetic speech signal comprises:
analyzing, via the one or more processors, the one or more biosignals and the synthetic speech signal to identify corresponding samples representative of individual words, syllables, phonemes, and/or other sounds of the target speech sequence; and
mapping, via the one or more processors, the samples of the synthetic speech signal to corresponding sample of the one or more biosignals.

10. The computer-implemented method of claim 9, wherein:
the one or more biosignals include an audio signal of the subject attempting to speak the target speech sequence; and
identifying the samples comprises segmenting, by the one or more processors, the audio signal based on breaks included in the audio signal.

11. The computer-implemented method of claim 9, wherein:
the one or more biosignals include one or more neural signals of the subject attempting to speak the target speech sequence; and
identifying the samples comprises identifying, by the one or more processors, dynamics of neural activity in the one or more neural signals.

12. The computer-implemented method of claim 9, wherein identifying the samples comprises:
inputting, via the one or more processors, the one or more biosignals into a second speech decoder to produce a second encoded speech signal;
analyzing, via the one or more processors, the second encoded speech signal to identify sample timing; and
applying, via the one or more processors, the identified sample timing to algin the synthetic speech signal with the one or more biosignals.

13. The computer-implemented method of claim 9, wherein identifying the samples comprises:
inputting, via the one or more processors, the one or more biosignals into a text prediction model configured to output a transcript and corresponding timings of speech represented by the one or more biosignals; and
identifying, via the one or more processors, the one or more biosignals based on the transcript and the corresponding timings.

14. The computer-implemented method of claim 9, wherein identifying the samples comprises:
inputting, via the one or more processors, the one or more biosignals into a time-series model configured to predict sample timings when each sample occurred; and
applying, via the one or more processors, the predicted sample timings to align the synthetic speech signal with the one or more biosignals.

15. The computer-implemented method of any one of claims 9 to 14, wherein mapping the samples comprises:
applying, via the one or more processors, a time-warping function to the samples to align durations of the samples with the corresponding segment of the one or more biosignals.

16. The computer-implemented method of any one of claims 9 to 15, wherein mapping the samples comprises:
applying, via the one or more processors, a para-linguistics matching function to the corresponding segment of the synthetic speech signal such that paralinguistic characteristics of the attempted speech represented by the one or more biosignals are exhibited by the synthetic speech signal.

17. The computer-implemented method of any one of claims 1 to 16, wherein the speech decoder includes a Transformer model.

18. The computer-implemented method of any one of claims 1 to 17, wherein the speech decoder includes a recurrent neural network (RNN).

19. The computer-implemented method of any one of claims 1 to 18, wherein the encoded speech signal is encoded in a format configured to be input into a vocoder that generates synthesized speech.

20. The computer-implemented method of any one of claims 1 to 19, wherein the encoded speech signal is encoded in a format configured to be input into a vocal tract model for synthesizing speech from articulatory kinematics and/or kinetics.

21. The computer-implemented method of any one of claims 1 to 20 wherein the subject is anarthric or dysarthric.

22. A system comprising:
one or more processors; and
a non-transitory memory storing processor-executable instructions that, when executed by the one or more processors, cause the system to perform the method of any one of claims 1 to 21.

23. A non-transitory computer-readable storage medium storing processor-executable instructions that, when executed by one or more processors, cause the one or more processors to perform the method of any one of claims 1 to 21.

24. A computer-implemented method for generating synthesized speech, the method comprising:
obtaining, via one or more processors, one or more biosignals from a subject attempting to speak;
inputting, via the one or more processors, the one or more biosignals into a speech decoder to obtain a frame of an encoded speech signal, wherein the speech decoder is trained using training data that includes aligned historic biosignals associated with the subject attempting to speak a target speech sequence and synthetic speech for the target speech sequence; and
generating, via the one or more processors, a frame of a synthesized speech signal by inputting the frame of the encoded speech signal into a speech synthesizer, wherein the speech decoder is configured to generate the frame of synthesized speech signal within about 10 milliseconds of obtaining the one or more biosignals.

25. The computer-implemented method of claim 24, wherein the speech decoder is configured to generate the frame of the synthesized speech signal within about 5 milliseconds of obtaining the one or more biosignals.

26. The computer-implemented method of claim 24 or 25, further comprising:
storing, via the one or more processors, the one or more biosignals in a buffer.

27. The computer-implemented method of claim 26, wherein inputting the one or more biosignals into the speech decoder further comprises:
obtaining, via the one or more processors, a segment of the one or more biosignals from the buffer; and
inputting, via the one or more processors, the segment of the one or more biosignals into the speech decoder.

28. The computer-implemented method of claim 27, wherein the segment is representative of at least 100 milliseconds of the one or more biosignals.

29. The computer-implemented method of claim 28, wherein the segment is representative of at least 200 milliseconds of the one or more biosignals.

30. The computer-implemented method of any one of claims 24 to 29, wherein the segment is representative of at least 600 milliseconds of the one or more biosignals.

31. The computer-implemented method of any one of claims 24 to 30, wherein the one or more biosignals are derived from data obtained from a sensor, wherein the sensor is selected from the group consisting of an intracortical electrode array, an electrocorticography (ECOG) sensor, a stereoelectroencephalography (sEEG) sensor, a surface electroencephalography (EEG) sensor, a functional near-infrared spectroscopy (fNIRS) sensor, a magnetoencephalography (MEG) sensor, an electromyography (EMG) sensor, an acoustic myography (AMG) sensor, an ultrasound sensor, a magnetomyography (MMG) sensor, a light detection and ranging (LiDAR) sensor, an infrared sensor, a video sensor, an audio sensor, a vibration sensor, a motion sensor, an inertial measurement unit (IMU), a piezoelectric sensor, a breathing sensor, another intracortical device capable of recording individual neurons, and any combination thereof.

32. The computer-implemented method of claim 31, wherein the sensor is coupled with a motor cortex of the subject.

33. The computer-implemented method of claim 31, wherein the sensor is coupled with an inferior frontal gyrus, a superior temporal gyrus, basal ganglia, a cerebellum, a brainstem, a thalamus, or a subthalamic nucleus of the subject.

34. The computer-implemented method of any one of claims 24 to 33, wherein the speech decoder includes a Transformer model.

35. The computer-implemented method of any one of claims 24 to 34, wherein the speech decoder includes a recurrent neural network (RNN).

36. The computer-implemented method of any one of claims 24 to 35, wherein the frame of the encoded speech signal is encoded in a format configured to be input into a vocoder that generates synthesized speech.

37. The computer-implemented method of any one of claims 24 to 35, wherein the encoded speech signal is encoded in a format configured to be input into a vocal tract model for synthesizing speech from articulatory kinematics and/or kinetics.

38. The computer-implemented method of any one of claims 24 to 37, wherein the subject is anarthric or dysarthric.

39. The computer-implemented method of any one of claims 24-28, further comprising:
outputting, via an audio output device, the frame of the synthesized speech signal.

40. A system comprising:
one or more processors; and
a non-transitory memory storing processor-executable instructions that, when executed by the one or more processors, cause the system to perform the method of any one of claims 24 to 39.

41. A non-transitory computer-readable storage medium storing processor-executable instructions that, when executed by one or more processors, cause the one or more processors to perform the method of any one of claims 24 to 39.
